(19) United States
(12) Patent Application Publication
(10) Pub. No.: US 2005/0147959 A1
(43) Pub. Date: Jul. 7, 2005

(54) Tissue analogs for in vitro testing and method of use therefor

(76) Inventors: Carmelita G. Frondoza, Woodstock, MD (US); David J. Fink, Baltimore, MD (US)

Correspondence Address: Armstrong Kratz Quintos Hanson & Brooks;Intellectual Property Law Offices, Suite 220, 502 Washington Avenue, Towson, MD, 21204, US

(21) Appl. No.: 10/508,497

(22) PCT Filed: Mar. 21, 2003
(86) PCT No.: PCT/US03/10105

Related U.S. Application Data
(60) Provisional application No. 60/367,682, filed on Mar. 25, 2002.

(51) Int. Cl. A01N 1/02; C12Q 1/00; C12N 5/08

(52) U.S. Cl. 435/2; 435/4; 435/366

(57) ABSTRACT

A test system and method are disclosed for using tissue analogs The method includes the following steps: (1) isolating the cells to be implanted from donor tissue; (2) seeding the cells onto a particulate microcarrier bead; (3) culturing the cells on the microcarriers to achieve an expansion in the number of cells; and (4) further culturing the cell-particle aggregates to form a tissue analog. The resulting tissue analog and test system may be used for use in screening drugs for diseases and pathological conditions, for testing for toxicity of chemical agents, or for genomic or proteomic screening. The tissue analogs may also be subjected to conditions that will induce disease-like conditions such that the resulting diseased tissue analogs may be used to screen for therapeutic drugs that modify the diseased tissue analog physiology or block progression of the diseased conditions. Kits may be developed for the purpose of conducting multiple tests in conventional multi-well plate systems.

### RELATED APPLICATIONS

This application is related to provisional application Ser. No. 60/367,682 filed Mar. 25, 2002 and U.S. patent application Ser. No. 09/825,632, filed Apr. 4, 2001.

### FIELD OF THE INVENTION

The herein disclosed invention finds applicability in the field of cell culture, as well as in the field of cell-based in vitro testing of biocompatibility, drug screening, genomics and proteomics.

### BACKGROUND

Cultured cells and tissue explants have been used extensively in vitro to study the biology, metabolism and function and many connective tissues including cartilage, bone, tendon, ligament, synovium, and meniscus, and many organs such as liver, kidney and pancreas.

For example, cultured cartilage cells (chondrocytes) and cartilage explants have been used extensively to study cartilage biology, metabolism and function. Isolated chondrocytes have been propagated in monolayer culture and in constrained environments such as in pellet culture or in three-dimensional scaffolds. These in vitro models have provided critical information on cartilage biology at the level of individual chondrocytes. However, these in vitro models are limited in providing sufficient numbers of chondrocytes that exhibit features of their original phenotype.

The inventors and their colleagues have developed a spinner bioreactor culture system in which chondrocytes proliferate and continue to produce extracellular matrix (ECM) components consisting of type II collagen and high molecular weight proteoglycans characteristic of hyaline cartilage. The cell-seeded microcarriers form cartilage tissue-like material [1]. The current invention is based on the observation that the newly formed “cartilage analog” responds to biological, chemical, physical and mechanical stimuli reminiscent of their cartilage counterpart. Responses to such stimuli are indicated by changes in proliferative capacity, phenotypic expression, synthesis of protein and proteoglycan products and other metabolic indices. Based on these observations, the inventors have shown that cartilage analogs offer a novel technology platform to screen for effects of various agents or factors on chondrocyte and cartilage biology. These cartilage analogs allow standardized screening of a wider variety of test agents than current non-standard tests by providing a more reproducible, reliable and plentiful supply of test material.

In a similar manner, analogs of bone or connective tissues may be grown by culturing appropriate differentiated cells on microcarrier beads, and relatively undifferentiated tissues such as the mesenchyme may be grown by culturing stem cells such as mesenchymal stem cells on microcarrier beads. The significance of such analogs is demonstrated below based on the use of cartilage analogs.

Cartilage Structure, Chemical Composition and Biomechanical Properties.

Articular cartilage is critically important in normal joint function. This avascular, aneural, 2-4 mm-thick tissue consists of a type II collagen framework interspersed with proteoglycans. The physico-chemical properties of cartilage allow it to serve as a resilient load-bearing material. Composed primarily of extracellular matrix and chondrocytes, articular cartilage provides the excellent low friction, lubrication and wear characteristics needed to meet the biomechanical requirements in the joint. In non-articulating sites such as the nose, chondrocytes produce cartilage that provides structural function. Chondrocytes comprise only about 5% of the cartilaginous tissue but they are responsible for the continual active synthesis and degradation of the cartilage extracellular matrix (ECM).

When damaged as a consequence of disease or mechanical injury, cartilage is unable to heal. This is attributed to the limited capacity of chondrocytes to proliferate and to the absence of blood supply in the tissue. The repair tissue consists of fibrocartilage, which differs in chemical structure from hyaline cartilage. Altered chemical composition compromises biomechanical function and eventually requires joint replacement. Cartilage deterioration is observed in certain diseases such as osteoarthritis, in conditions of reduced load such as prolonged bed rest or immobilization; and under conditions of excessive load. Thus, maintenance of physiologic biomechanical environment is required for promoting normal joint function.

Articular cartilage has a high water content ranging from 65% to 80% of its wet weight. The high frictional resistance against flow and the water pressure in articular cartilage allow this tissue to act as a cushion and support heavy loads. The flow of water through the cartilaginous tissue also provides a vehicle for transporting nutrients. In addition, the flow of water serves as a source of lubricant in the joint. Water is stored in the extracellular matrix by hydrophilic proteoglycans. The collagen network comprises more than 50% of the cartilage dry weight and 90-95% of this collagenous network is composed of type II collagen. Type II collagen provides the tensile strength of cartilage, while other minor collagens, such as types VI, IX, X, and XI, contribute to the stability of the intricately cross-linked lattice.

Trapped within the collagen network of articular cartilage are proteoglycans, which primarily exist in large aggregating complexes consisting of a protein core (aggrecan) to which the glycosaminoglycans chondroitin sulfate and keratan sulfate are attached. These large aggrecan complexes facilitate charge immobilization within the confines of the collagen network. Other proteoglycans present in articular cartilage such as decorin and biglycan also contribute towards maintenance of matrix integrity.

The physico-chemical and structure-function properties of articular cartilage have been extensively studied and modeled. For example, a two-phase model has been proposed in which a solid phase represented by the cartilage matrix is attached to the subchondral bone while a fluid phase flows freely through the tissue. Under normal conditions, articular cartilage can withstand forces of considerable magnitude. While standing, the pressure applied on articular cartilage in the hip is estimated to be 0.7 MPa; stresses may approach 20 MPa in standing up from a sitting position. Chondrocytes are responsible for detecting, processing and responding to such mechanical signals in the tissue.

Production and breakdown of the cartilage ECM are influenced by a wide variety of bioactive molecules, including growth factors such as transforming growth factor-β (TGF-β) and cytokines such as IL-1β and TNF-α. TGF-β is a potent regulator of chondrocyte proliferation, differentiation and ECM accumulatio that regulates the metabolism of proteoglycans. Cytokines are produced by a wide variety of cells and have been documented to regulate inflammatory and immune responses. The mechanism of proteoglycan degradation induced by cytokines involves induction of proteolysis by induction of metalloproteinases (MMPs). Breakdown of the protein core results in the release of hyaluronate and proteoglycan fragments. The chemical nature of proteoglycan degradation products induced by cytokines appears to be the same as the degradation products resulting from normal catabolic processes. Based on this finding, cytokines appear to mediate degradation of proteoglycans by accelerating catabolic rates. In arthritis, IL-1β and TNF-α are the major cytokines mediating the breakdown of cartilage.

Known as key mediators of inflammation, prostaglandins are also associated with the regulation of cartilage breakdown. The biosynthesis of prostaglandins is regulated by the enzyme cyclooxygenase (COX). There are two enzyme isoforms: COX-1 and COX-2. The isoforms have approximately 60% amino acid homology with similar tertiary structures but different active sites. They also differ in their patterns of tissue and cellular distribution. COX-1 is constitutive and is widely distributed throughout the body. COX-2 is expressed by inflammatory cells and by chondrocytes. In the inflammatory response of OA chondrocytes, COX-2 expression is increased and appears to be a major determinant of prostaglandin E₂ activity. COX-2 expression also appears to be responsive to IL-1 β and TNF-α stimulation.

Degradative enzymes are critical in the control of cartilage turnover. These proteinases function as regulatory molecules by participating in enzyme cascades and by processing matrix proteins, cytokines and growth factors to produce molecules with enhanced or reduced biological effects. These degradative enzymes include MMPs that are expressed as inactive zymogens, which have pro-domains that are first dissociated from the catalytic domain prior to activation of the enzyme. Tissue inhibitors of metalloproteinases (TIMPs) are natural inhibitors of MMPs. The dynamic balance between the MMPs and TIMPs play a crucial role in the maintaining the integrity of articular cartilage. In OA, the dynamic balance between the MMPs and TIMPs is disrupted resulting in increased breakdown of cartilage.

Chondrocyte Cultures as Models for Evaluating Cartilage Responses.

Chondrocyte cultures have been studied extensively since the discovery that these viable cells can be retrieved through enzymatic digestion. One of the most commonly used techniques for chondrocyte propagation is monolayer culture, wherein cells are seeded to spread out into a single layer. Although these cells multiply in monolayer culture, they also undergo phenotypic changes and assume “fibroblastoid” morphologic and biochemical characteristics, a process commonly called “dedifferentiation”. They shift from making type II collagen to type I and decrease their synthesis of high-molecular-weight proteoglycans. Cell spreading in monolayer culture destabilizes the chondrocytic phenotype and favors proliferation. Approaches to constrain cell spreading and to simulate the three-dimensional environment in cartilage have been taken to help retain the chondrocyte phenotype. To minimize dedifferentiation, other strategies have been used such as culturing chondrocytes at high density in pellet culture. Alternatively, chondrocytes have been propagated in alginate beads or in three-dimensional matrices. These in vitro chondrocyte models have provided valuable information on chondrocyte metabolism and function including identification of the effects of biologic, chemical, pharmacological, physical and mechanical factors on these cells. The growth-differentiation factor TGF-β is an example of a cytokine that has been widely examined using chondrocyte cultures [2].

Chondroycte culture models have also provided evidence that physical factors such as oxygen tension and mechanical forces influence the biology of cartilage and chondrocytes. Investigations in man, in animals, and using in vitro models demonstrate that mechanical signals are detected, processed, and transduced by chondrocytes, which recognize their biomechanical environment as they are subjected to load, compression, stretch, and shear. Detection of mechanical signals triggers a series of molecular events that determine whether chondrocytes divide, differentiate, or continue to maintain their phenotype. Several in vitro models have been used to study the effect of mechanical stress on chondrocytes: (a) Static or dynamic compression ; (b) Cyclic strain; and (c) Fluid induced-shear. Extensive literature from these in vitro tissue culture models indicates that chondrocytes sense and respond to mechanical signals. Exposure of chondrocytes to compression, cyclic strain, or fluid-induced shear results in altered proliferation rates and metabolism.

In Vitro Cartilage Analog Test System.

Clusters of tissue-producing cells, cultured on microcarrier beads to produce a tissue analog, express responses similar to intact tissue when exposed to drugs, toxins, physical conditions such as mechanical stresses, or electrical or magnetic fields, or combinations of such agents and conditions. Applications may include screening drugs to treat diseases and pathological conditions of cartilage, toxicological testing of drug candidates or other toxicants, and for use as cartilage substitutes in genomic and proteomic screening. For example, chondrocytes may be cultured on collagen microcarrier beads to produce cartilage analogs, which then may be used as a test system to determine the effects of drugs such as ibuprofen, aspirin, or COX-2 inhibitors; growth factors such as PDGF, FGF, BMPs, TGF-β; or cytokines such as TNF-α, IL-1α or IL-1β. Other tissue applications may involve, for example, the use of osteoblastic cells to produce bone analogs and pluripotent cells such as mesenchymal stem cells or embryonic stem cells to produce latent tissues analogs that might be converted to differentiated tissue analogs when cultured in appropriate differentiation conditions. Tissue analogs produced by these methods might be further treated to induce disease-like responses. For example, cartilage analogs might be exposed to collagenases and cytokines such as TNF-α or IL-1 to induce an arthritic condition or response.

In vitro testing using microcarrier-based tissue analogs may be conducted in standard culture vessels including spinner culture systems, in multi-well microtiter plate systems, or in novel culture systems designed to minimize the amount of tissue analog tested. Microtiter plate systems having 24, 96, 384 or more wells in each plate are advantageous because they may be conveniently processed and monitored by standard laboratory equipment constructed for such purposes as dispensing fluid reagents, independently washing the wells, monitoring the wells by optical or fluorescent instrumentation, removing fluid samples from the wells during the culture and after the culture is terminated, and mixing the plates on rocking or rotary plate mixers. Furthermore, cells may added to the wells and cryopreserved therein in order to provide kits suitable for transporting frozen pre-determined quantities of the tissue analog.

Advantages of the Tissue Analog Test System.

In the past decade, considerable effort has been directed to the use of tissue or organ analogs suitable for use as in vitro test systems for use in toxicology testing and drug screening, with the objective of minimizing initial testing in live animal models. The use of in vitro test systems offers the potential to reduce the number of animals used in testing, to standardize test systems by providing well-characterized test conditions, and to reduce the cost of the testing of libraries of potentially therapeutic and/or toxic compounds. Automation of test systems that use small analogs of cartilage could permit the screening of libraries of drugs using conventional high-through-put screening systems modified to accommodate cell culture conditions. Because each analog particle contains all the properties of the tissue analog, test systems can be miniaturized to incorporate testing of a single particle. Tests using tissue analogs are also simpler and more standardized than in vitro tests that employ explant culture of whole or partial thickness pieces of cartilage. Culturing of tissue analogs to produce large batches of the analogs also permit in vitro testing in longitudinal studies having multiple time points using cells of identical origin and at sufficient scale to permit macroscopic evaluation or analysis and the development of differential cDNA libraries, for example.

In vitro chondrocyte culture models have been used extensively to evaluate tissue responses to a variety of agents including pharmacologic and biological factors. These models have the advantage of being easily accessible, utilizing relatively small number of cells, and permitting analyses at reasonable cost and time. Use of in vitro models also reduces the number of animals used in preclinical testing. These models provide critical insights into the cellular-molecular mechanisms involved and the potential mode of action of the agent being tested. However, use of cartilage models is time-consuming and subject to great variability. No standard in vitro model exists for cartilage and each experiment requires the researcher to source, isolate and culture chondrocytes or cartilage explants. Often, human tissue is not accessible, so cartilage from bovine or other large animals are frequently substituted for human tissues.

Therefore, advantages of the proposed in vitro test system include:
1. Standardization of the methods of production and storage in order to provide reproducible tissue analogs.
2. Potential for producing large numbers of cartilage analogs, permitting high thoughput screening of large numbers of drug candidates or toxic agents.
3. Flexibility of the microcarrier format, enabling use of small amounts of tissue analogs in multi-well plate formats or other formats designed to accommodate bead-based reagents.
4. Savings of time and resources resulting from individual researchers no longer needing to procure, process and culture cells or explants from human or animal tissues.
5. Availability of human tissue analogs so that studies of surrogate animal tissues are no longer needed.
6. Observed similarity of cartilage analogs to known cartilage-modifying drugs in preliminary studies.

Typical settings for use of the tissue analogs will be academic or industrial research laboratories performing disease-related research, screening drug candidates, or conducting genomic or proteomic research of tissues or diseased tissues.

The major limitation of this approach is that a cartilage analog consisting of isolated chondocytes is examined apart from the normal physiologic environment. Moreover, the cartilage analog may display features characteristic of hyaline cartilage phenotype but some markers inherent to the original tissue may not be detectable. However, the total systemic-organ interaction is not captured in the tissue analog, which is the case for all in vitro models. Therefore, use of these models may be generally limited to the initial stages of screening. Tissue analogs of the type proposed also may be difficult to adapt to in vitro testing involving dynamic loading.

No documented standard in vitro models are available to replace the use of in vivo animal models. Cartilage explants are widely used, especially for investigations of mechanical effects on cartilage physiology and function. These explant models are especially time-intensive and are usually based on surrogate animal cartilage. An in vitro “neocartilage” construct was described [3,4] that may have utility for drug screening and toxicology testing in the manner of that proposed in this application. In this model system, chondrocytes are cultured in the wells of multi-well plates to produce a cartilage-like tissue for therapeutic or in vitro applications. The inventors expect that in vitro test systems based on the “neocartilage” procedure may represent a less flexible format and, for example, be more difficult to use in miniaturized test systems than the microcarrier bead-based analogs disclosed in the current invention. Production costs for “cartilage analogs” should also be lower if the analogs are prepared in large batches instead of cultured in individual wells.

Significance.

The need for in vitro test products of this type is recognized by the National Institutes of Health in its Omnibus SBIR/STTR Solicitation for 2002, specifically by the National Institute of Arthritis and Musculoskeletal and Skin Diseases, the National Institute of Dental and Craniofacial Research, and the National Institute of Environmental Health Sciences.

### SUMMARY OF THE INVENTION

A method is disclosed for fabricating tissue analogs for use in in vitro test systems for screening drugs for diseases and related pathological conditions. The method includes the following steps: (1) isolating the cells to be implanted from donor tissue; (2) seeding the cells onto a particulate microcarrier bead; (3) culturing the cells on the microcarriers to achieve an expansion in the number of cells; and (4) further culturing the cell-particle aggregates to form a tissue analog. The resulting analogs may be used for in vitro test systems, for example, for screening drugs for acute toxicity to healthy tissue. The tissue analogs may also be subjected to conditions that will induce, for example, disease-like conditions such that the resulting diseased tissue analogs may be used to screen for therapeutic drugs that modify the diseased analog physiology or block progression of the disease conditions.

### DESCRIPTION OF DRAWINGS

FIG. 1. A two-component multi-well culture plate system.

FIG. 2. Procedure for conducting a test in a two-component multi-well culture plate system.

FIG. 3. Photomicrographs of cells cultured on microcarrier beads and in monolayer culture.

FIG. 4. RT-PCR results for phenotypic gene expression of cultured chondrocytes.

FIG. 5. Gene expression of types IX and XI collagens by cultured chondrocytes analyzed by RT-PCR.

FIG. 6. Photomicrograph showing early stages of aggregation of chondrocyte-seeded microcarriers on day 7 in spinner culture.

FIG. 7. Photomicrograph showing formation of cartilage-like tissue produced on day 60 in spinner culture.

FIG. 8. Results of a test using cartilage analogs cultured in spinner reactors to determine the effect of a proposed chodroprotective drug.

FIG. 9. Photomicrograph of cartilage analogs from a test in a 96-well assay format.

FIG. 10. Results from a test in a 96-well assay format: effect of FGF-9 on cell growth in cartilage analogs.

FIG. 11. Results from a test in a 96-well assay format: effect of FGF-9 on total RNA in cartilage analogs.

FIG. 12. Results from a test in a 96-well assay format: effect of FGF-9 on gene expression by cartilage analogs.

FIG. 13. Results from a test in a 96-well assay format: effect of FGF-9 on total proteoglycan production by cartilage analogs.

### DETAILED DESCRIPTION OF DRAWINGS

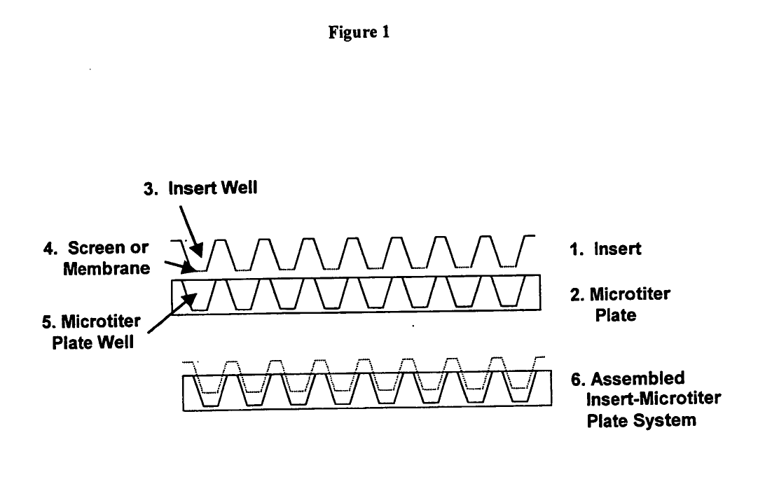

FIG. 1. A two-component multi-well culture plate system. A standard microtiter plate (2) is fitted with an insert component (1) having molded extensions or wells (3) that fit into each well of the multi-well culture plate (5). At the bottom of each extension is placed a porous screen or semipermeable membrane (4) that effectively covers the bottom of the well of the multi-well culture plate.

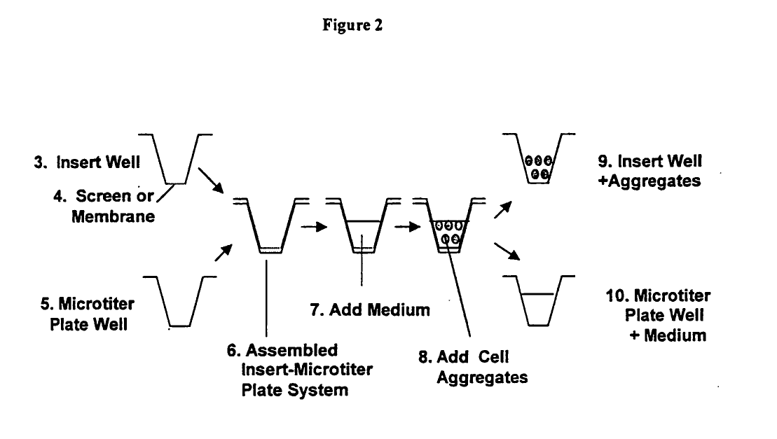

FIG. 2. Procedure for conducting a test in a two-component multi-well culture plate system. Using the two-component system of FIG. 1, an assay is initiated using the assembled two-component system (6) by dispensing medium and test substances into the wells of the inserts (7), dispensing tissue analogs (8), and culturing these components for a desired period under standard culture conditions. At the end of the culture period, separation of the insert from the microtiter plate results in drainage of the fluid components of the culture medium into the microtiter well, thereby rapidly separating the tissue analogs (9) from the media (10) in each well without requiring a centrifugation or sedimentation step. The tissue analogs also may be conveniently washed in the wells of the insert. Using this system, the amount of tissue analog required for an assay may be dispensed into the insert and cryopreserved therein in order to facilitate the use of the inserts at a later time and to preserve the analogs in a standardized stage of development.

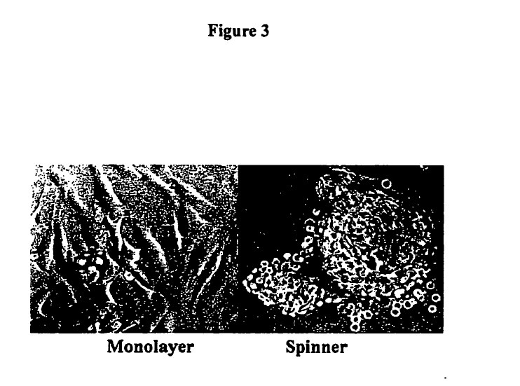

FIG. 3. Photomicrographs of cells cultured on microcarrier beads and in monolayer culture. Chondrocytes proliferated on the surface of the beads, attaining 90% confluence in about eight days. Cell-coated beads (right panel) revealed the presence of spherical chondrocytes. These cells have abundant cytoplasm and eccentric nuclei. The deposition of extracellular matrix-like material is evident around the beads and, more prominently, in the regions of cell-to-cell adhesion. In contrast, matched chondrocytes in monolayer culture appeared elongated and spindle-shaped (left panel).

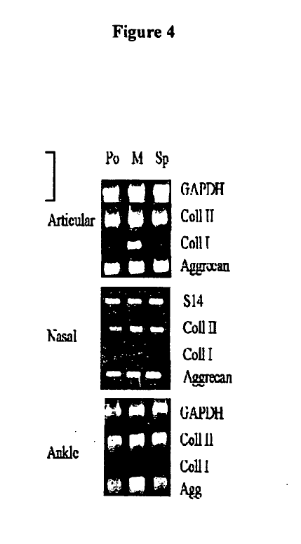

FIG. 4. RT-PCR results for phenotypic gene expression of cultured chondrocytes. Tissue samples were retrieved from knee (articular), nasal septum and ankle cartilage. Cells were cultured using the monolayer and spinner culture methods. RT-PCR was performed using probes for types I and II collagen and aggrecan as phenotypic markers. RT-PCR demonstration of gene expression displayed by chondrocytes is shown in the right panel. Samples were obtained directly from the tissue (Po), chondrocytes propagated in mololayer culture for one passage about 2 weeks (M) and chondrocytes propagated about 2 weeks in microcarreir spinner culture (Sp). RT-PCR indicate that cells isolated from articular knee cartilage of OA patients, ankle cartilage and nasal septal cartilage, and propagated in spinner culture, maintained expression of type II collagen and aggrecan, but type I collagen was down-regulated.

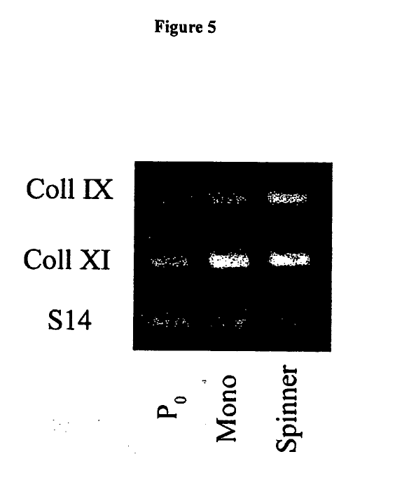

FIG. 5. Gene expression of types IX and XI collagens by cultured chondrocytes analyzed by RT-PCR. Gene expression was analyzed in articular chondrocytes obtained directly from knee cartilage (Po), chondrocytes propagated in monolayer culture for one passage for about 2 weeks, and chondrocytes propagated in microcarrier spinner culture for about 2 weeks (Sp). The RT-PC profiles show that expression of type IX collagen is enhanced when chondrocytes were propagated in microcarrier spinner culture.

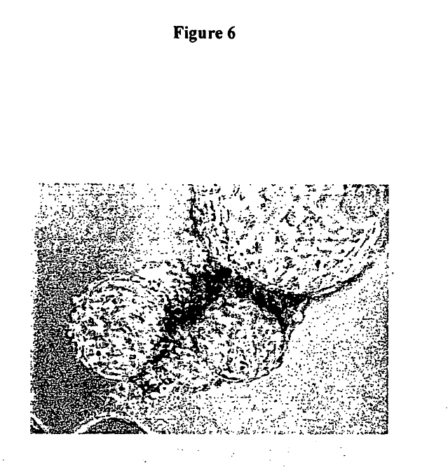

FIG. 6. Photomicrograph showing early stages of aggregation of chondrocyte-seeded microcarriers on day 7 in spinner culture.

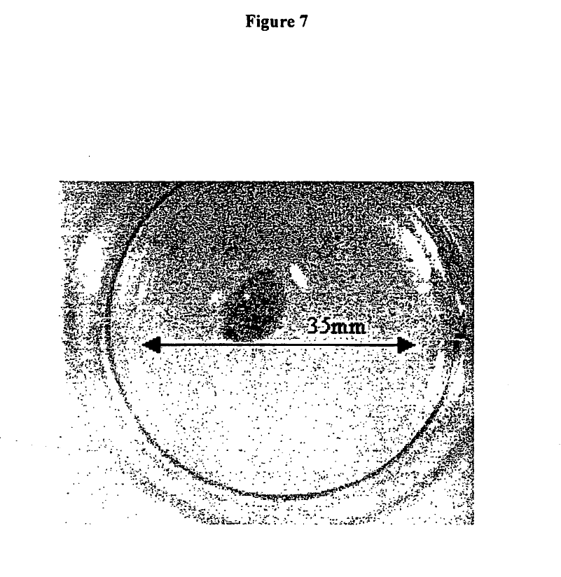

FIG. 7. Photomicrograph showing formation of cartilage-like tissue produced on day 60 in spinner culture.

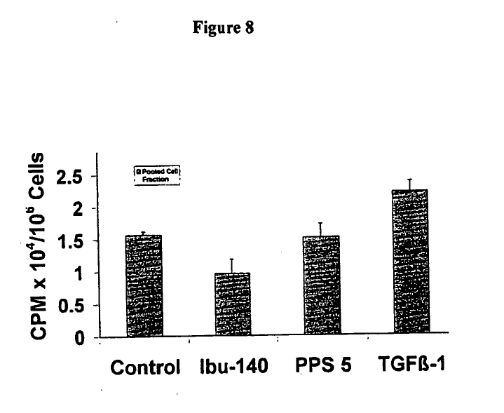

FIG. 8. Results of a test using cartilage analogs cultured in spinner reactors to determine the effect of a proposed chodroprotective drug. Human chondrocytes from donors undergoing nasal septal defect surgery were isolated by enzymatic digestion, seeded onto microcarriers, and incubated in spinner flasks until the cells reached confluency at about 14 days. Equivalent aliquots of these cartilage analogs were transferred to four 25-ml spinner flasks. Aliquots of control medium (no additive), or medium containing human platelet TGF-β₁, Ibuprofen or a proposed chondroprotective drug (PPS5) were added to duplicate flasks. 24 hours prior to terminating the cultures, ³⁵S—Na₂SO₄ was added to each flask to measure incorporation into proteoglycans during the last 24 hours of culture. De novo proteoglycan synthesis was used as a marker for chondrocytic activity as shown. Ibuprofen decreased proteoglycan synthesis, TGF-β₁ enhanced protoglycan synthesis and the test drug PPS5 did not show any significant effect on proteoglycan synthesis during the assay period compared to the control.

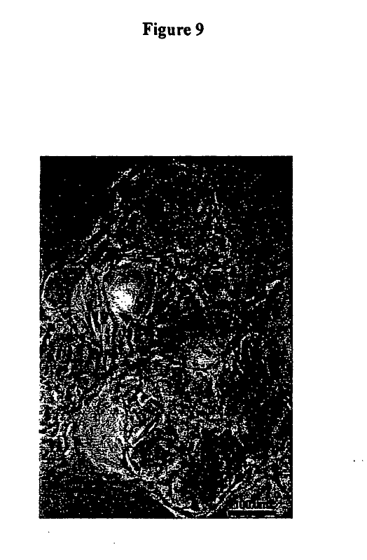

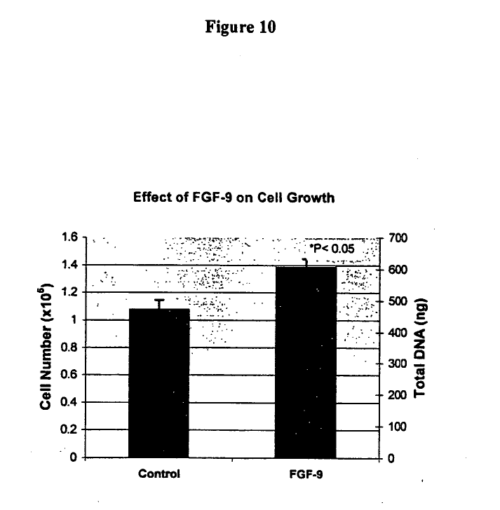

FIG. 9. Photomicrograph of cartilage analogs from a test in a 96-well assay format. Nasal chondrocytes were seeded in a spinner flask onto Cellagen® microcarrier beads and cultured in a spinner flask for about 10 days. The cell-seeded microcarriers were then sedimented and the volume of media was reduced. Aliquots of the cartilage analogs shown were transferred into a total of 30 wells in a 96-well plate. Control media alone, or media containing FGF-9 and heparin, was added to the wells, and the plate was incubated for 24 hours. Results of the test were analyzed and compared to controls as shown in FIGS. 10 (cell growth), 11 (total RNA), 12 (gene expression), and 13 proteoglycan production.

FIG. 10. Results from a test in a 96-well assay format: effect of FGF-9 on cell growth in cartilage analogs.

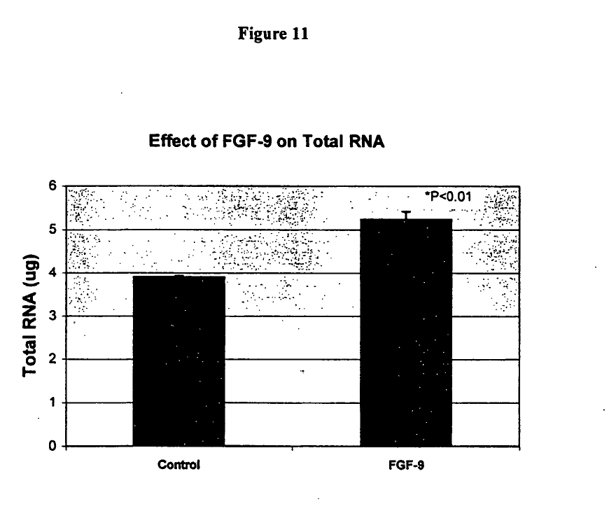

FIG. 11. Results from a test in a 96-well assay format: effect of FGF-9 on total RNA in cartilage analogs.

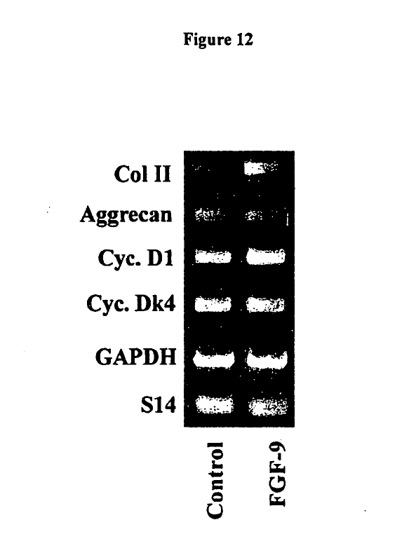

FIG. 12. Results from a test in a 96-well assay format: effect of FGF-9 on gene expression by cartilage analogs.

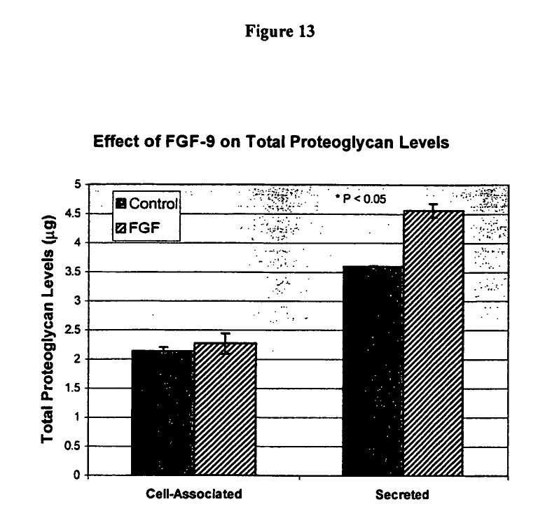

FIG. 13. Results from a test in a 96-well assay format: effect of FGF-9 on proteoglycan production by cartilage analogs.

### DETAILED DESCRIPTION OF THE INVENTION

A general method for fabricating tissue analogs is described for use for in vitro test systems for screening drugs for diseases and pathological conditions of cartilage. For example, a method for fabricating cartilage tissue analogs is described for use as in vitro test systems for screening drugs for diseases and pathological conditions of cartilage. The method includes the following steps: (1) isolating chondrocyte or chondrocyte precursor cells to from donor tissue; (2) seeding the cells onto a particulate microcarrier material; (3) culturing the cells on the microcarriers to achieve an expansion in the number of cells; and (4) further culturing the cell-particle aggregates to form a tissue analog of articular cartilage. The resulting cartilage analogs may be used as in vitro test systems, for example, for screening anti-arthritis drugs for acute toxicity to healthy cartilage tissue. The analogs may also be subjected to conditions that will induce, for example, arthritic-like conditions such that the resulting arthritic cartilage analogs may be used to screen for therapeutic drugs that modify the arthritic analog physiology. In general, the method for producing tissue analogs includes the following steps:
1. isolating the cells to be used to produce the tissue analog from donor tissue;
2. seeding the cells onto a particulate material;
3. culturing the cells on the particles to achieve an expansion in the number of cells; and
4. further culturing the cell-particle aggregates to form a tissue analog.

A possible additional step may include the treatment of the tissue analogs to conditions whereby they exhibit one or more attributes that mimic a disease of that tissue.
5. the tissue analogs are further cultured in the presence of disease-inducing agents such as enzymes, drugs, hormones, cytokines, tissue extracts, fluids from diseased tissue or joints, or combinations of such agents.

In a specific example of this method, chondrocytes are cultured on microcarrier particles to produce cartilage analogs after 7-14 days of culture. Chondrocytes propagated in spinner culture on biopolymer beads eventually aggregate into cartilage-like masses. These materials would be suitable for use for in vitro toxicology or drug screening test systems [1].

Methods for preparing cartilage analogs are further described in U.S. patent application Ser. No. 09/825,632, filed Apr. 4, 2001 for “Method for Fabricating Cell-containing Implants”; Frondoza et al., Inventors (reference Docket No. 20303-PA). However, this application considers only the therapeutic use of the tissue analogs by implantation.

It is anticipated in this invention that clusters of tissue-producing cells, cultured on microcarrier beads to produce a tissue analog may express responses similar to intact tissue when exposed to drugs, toxins, or physical conditions such as mechanical stresses, or electrical or magnetic fields or combinations of such agents or conditions. For example, chondrocytes may be cultured on collagen microcarrier beads to produce a cartilage analog, which may be used as a test system to determine the effects of drugs such as ibuprofen, aspirin, or COX-2 inhibitors; growth factors such as PDGF, FGF, BMPs, TGF-β; or cytokines such as TNF-α, IL-1α or IL-1β. In a second example, osteoblastic cells may be cultured on collagen microcarrier beads to produce a bone analog, which may be used as a test system to determine the effects of drugs such as ibuprofen, aspirin, or COX-2 inhibitors; hormones such as estrogen or testosterone; growth factors such as PDGF, FGF, BMPs, TGF-β; or cytokines such as TNF-α, IL-1α or IL-1β. In a third example, fibroblastic cells may be cultured on collagen microcarrier beads to produce a soft tissue or skin analog, which may be used as a test system to determine the effects of drugs, hormones, growth factors, or cytokines.

It is also anticipated that pluripotent cells such as mesenchymal stem cells or embryonic stem cells may be cultured on microcarrier beads to produce latent tissues analogs that might be converted to differentiated tissue analogs when cultured in the presence of appropriate differentiation factors such as BMPs, TGF-β, or dexamethasone, or combinations of such differentiating factors.

It is further anticipated that tissue analogs produced by these methods might be further treated to induce disease-like responses. For example, cartilage analogs might be exposed to collagenases and cytokines such as TNF-α or IL-1 to induce an arthritic condition or response. Alternatively, cartilage analogs might be exposed to diseased fluids or tissues from diseased joints in order to produce analogs of the diseased tissues. For example, synovial fluid from arthritic joints may be used, perhaps in combinations with other factors such as collagenases and/or cytokines such as TNF-α or IL-1, to produce an arthritic condition in the analogs.

Tissue analogs suitable for use as in vitro test systems may also be produced by further culturing the cell-microcarrier analogs in a molding device to prepare a standardized shape and size to accommodate the automated test system as described in U.S. patent application Ser. No. 09/825,632.

In vitro toxicology or drug screening tests may be performed using tissue analogs by the following steps:
1. preparing tissue or diseased-tissue analogs using the methods described above;
2. placing the analog(s) in an appropriate culture medium in a device suitable for continued culturing of the analog(s);
3. introducing the desired test substance (drug candidate, potential toxin, tissue extract, body fluid, enzyme, etc.) into the culture medium; and
4. analyzing and recording the effect of the test substance on the analog(s) continuously, periodically or after a specific period of time by monitoring characteristic analog outcome measures.

Outcome measures may include, but are not limited to: analog size, shape, density, color or opacity; changes in cell number; cell death or proliferation; changes in secreted materials such as cytokines, growth factors, hormones or extracellular matrix components including collagens, proteoglycans or glycosaminoglycans; genetic markers that are up- or down-regulated during the culture period such as genes for receptors, cytokines, integrins, extracellular matrix molecules, or enzymes; and cell-surface molecules including integrins and receptors.

The microcarrier may be inorganic or organic materials suitable for maintaining seeded cells in culture. Inorganic materials include, for example: calcium phosphates, calcium carbonates, calcium sulfates, glasses or combinations of these materials. Organic materials may include, for example: biopolymers such as collagen, gelatin, chitin, chitosan or chitosan derivatives, fibrin, dextran, agarose, or calcium alginate; particles of tissues such as bone or demineralized bone, cartilage, tendon, ligament, fascia, intestinal mucosa or other connective tissues; or chemically modified derivatives of these materials. Organic materials might also include synthetic polymeric materials, including, for example: polylactic acid, polyglycolic acid or combinations of the two, polyurethanes, polycarbonates, polyacrylates, or polypeptides.

The microcarrier material may also be used as a carrier for bioactive peptides (growth factors, cytokines, integrins, adhesion molecules, etc.), either to be released from the interior of the microcarriers or coated onto the surface of the particles, in order to improve cell adhesion or expansion, expression of phenotypic extracellular matrix, or other characteristics favorable to the production of a suitable tissue analog.

Microcarriers may be in the size range of 50-1000 μm, with the preferred size predominately in the range of 100-300 μm.

Cells useful in producing tissue analogs include, but are not limited to, differentiated cells including chondrocytes; osteoblasts; myoblasts; fibroblasts derived from skin, tendon, ligament, meniscus, disk or any other connective tissue; mesenchymal stem cells; pluripotent stem cells derived from bone marrow stroma, muscle, skin, fat, periosteum, perichondrium or other stem cell-containing tissue; embryonic stem cells; or combinations of these cells that may be seeded onto the microcarrier. Certain analogs for organs may also be produced by these methods, including liver, kidney and pancreatic analogs.

It is anticipated that in vitro testing using tissue analogs may be conducted in standard culture vessels including spinner culture systems or in multi-well microtiter plate systems. Microtiter plate systems having 24, 96, 384 or more wells in each plate are advantageous because they may be conveniently processed and monitored by standard laboratory equipment constructed for such purposes as dispensing fluid reagents, independently washing the wells, monitoring the wells by optical or fluorescent instrumentation, removing fluid samples from the wells during the culture and after the culture is terminated, and mixing the plates on rocking or rotary plate mixers. Furthermore, cells may added to the wells and cryopreserved therein in order to provide kits suitable for transporting frozen pre-determined quantities of the tissue analog.

A two-component microtiter plate system may also be configured as shown in FIG. 1. A standard microtiter plate (2) is fitted with an insert component (1) having molded extensions or wells (3) that fit snugly into each well of the microtiter plate (5). At the bottom of each extension is placed a porous screen or semipermeable membrane (4) that effectively covers the bottom of the well of the microtiter plate. The screen or membrane allows for rapid diffusion or convection of solutes or fluids between the two chambers, but retains tissue analogs in the insert well. An assay (FIG. 2) is initiated using the assembled two-component system by dispensing tissue analogs, medium and test substances into the well of the insert and culturing these components for a desired period under standard culture conditions. At the end of the culture period, separation of the insert from the microtiter plate results in drainage of the fluid components of the culture medium into the microtiter well, thereby rapidly separating the tissue analogs from the media in each well without requiring a centrifugation or sedimentation step. The tissue analogs also may be conveniently washed in the wells of the insert. Using this system, the amount of tissue analog required for an assay may be dispensed into the insert and cryopreserved therein in order to facilitate the use of the inserts at a later time and to preserve the analogs in a standardized stage of development.

It is further anticipated that many outcome measures may be used to determine the results of in vitro tests using tissue analogs, including but not limited to the following:

Assay for Cell Viability and Proliferative Capacity.

Cell viability and proliferative capaciaty can be determined by the live-dead assay, and ³H-thymidine (1 μCi) incorporation can be used to indicate the proliferative capacity of cells. The radiolabel will be added to each well at desired time point of incubation. Radioactivity will be determined by liquid scintillation counting.

Proteoglycan Synthesis by ³⁵SO₄ Incorporation.

De novo proteoglycan synthesis will be assessed by ³⁵SO₄ incorporation and subsequent export into the media. The supernatant containing newly synthesized secreted products and the chondrocyte-microcarrier construct bound products of ³⁵SO₄-pulsed proteoglycans can be dtermined. Proteoglycans of molecular weight (MW) greater than 10,000 will be identified chromatographic and electrophoretic techniques. The size of the newly synthesized proteoglycans can be prepared for SDS-PAGE and run on a 9% acrylamide gel under reducing and dissociating conditions. The ³⁵SO₄-labeled band enters the 9% acrylamide gel and migrates less than 1 mm from the origin, indicate the presence of a large molecule(s).

RNA Extraction and Analysis by Reverse Transcriptase-Polymerse Chain Reaction (RT-PCR).

Cells in the construct can be lysed and total RNA extracted with Trizol© (Life Technologies™, Rockville, Md.). Equal amounts (1 μg) of total RNA will be subjected to reverse transcription into cDNA at 42° C. for one hour with oligo (dt₁₈) primers. The transcripts will then be amplified by RT-PCR. Sample cDNA will also be amplified for housekeeping gene such as rRNA subunit S14 for controls. Primers for collagens, proteoglycans, and for other markers can be used to generate the products for analyses. Primers for other products such as cytokines (TNF-α), enzymes (COX-2, iNOS) may be used.

Cytological-Immunocytochemical Analysis of Products such as Collagens and Proteoglycans.

Cells retrieved from the constructs by enzyme disaggregation can be fixed and stained with either hematoxylin-eosin (H&E) or with monospecific antibodies against different collagen types and proteoglycans such as: chondroitin-4-sulfate, keratan or Aggrecan. Immunostaining for the marker or product of interest will be visualized using immunoperoxidase technique with a substrate such as diaminobenzidine which will yield a brownish color. Cell preparations will be counterstained with 0.5% toluidine blue.

Western Blots.

Cells from the chondrocyte-microcarrier constructs can be resuspended in lysis buffer containing protease inhibitors. Protein concentrations can be determined and total cell extracts can be electrophoresed on 7% SDS-polyacrylamide gel overlaid with stacking gel. The proteins will be transferred from gel to nitrocellulose paper to immunolocalize the product using monospecific antibodies. To visualize the bands, the membrane blot can be exposed to BioMax Light Film (VWR, NJ).

ELISA Assays for Products such as TNF-α and IL-1β.

The levels of TNF-α and IL-1β can be quantified by sandwich enzyme-linked immunosorbent assay. Concentrations will be determined using an ELISA reader.

Determination of Enzyme Products such as TIMP and MMP-1.

Enzymatic products such as TIMP and MMP-1 can be assessed by serially diluting media samples and anzying enzymatic activity using commercially available kits based on an enzyme linked immunoasssay.

Microarray Profiles.

Gene expression can be analyzed following exposure of the chondrocyte-microcarrier constructs to the test agents using the microarray technology.

### EXAMPLE 1

### Preparation of Cartilage Analogs

Human nasal septum chondrocytes were isolated by collagenase digestion from tissue discarded following deviated septum reconstruction. The cells were seeded at 4×10³/cm² in monolayer culture and propagated in HY medium (Hank's Balanced Salt Solution, HBSS+10% Fetal Calf Serum) until nearly confluent (about 2 weeks). Cells were harvested by trypsinization and seeded onto collagen microcarriers (4×10³ cells/cm² on Cellagen® beads, ICN, Cleveland, Ohio). The cultures (P3) were incubated for 5 to 15 days at 37° C., 5% CO₂. These chondrocyte-microcarrier-ECM aggregates are referred to as “cartilage analogs”.

Chondrocytes directly propagated in microcarrier spinner culture (primary cultures) retain their expression of type II collagen and aggrecan [1]. In contrast, matched chondrocytes propagated in monolayer culture decrease production of type II collagen while increasing their production of type I collagen. Furthermore, dedifferentiated chondrocytes that have been in monolayer culture for as long as six passages (about 3 months) resume production of type II collagen and suppress production of type I collagen.

In most cultures, human chondrocytes attach and proliferate on microcarrier spinner culture with a doubling time of about 2 to 3 days. The mean viability is greater than 95% at each cell harvesting, and there is no noticeable decrease in proliferative ability, as determined by cell counting, or viability, as determined by the vital dye exclusion assay. For example, chondrocytes seeded in microcarrier spinner culture attach readily to collagen (Cellagen™) microcarrier beads during the initial intermittent stirring phase of the seeding process. By day five, most microcarriers exhibit adherent cells on their surfaces. Chondrocytes proliferate on the surface of the beads, attaining 90% confluence in about eight days. Cell-coated beads (FIG. 3, right panel) reveal the presence of spherical chondrocytes. These cells have abundant cytoplasm and eccentric nuclei. The deposition of extracellular matrix-like material is evident around the beads and, more prominently, in the regions of cell-to-cell adhesion. In contrast, matched chondrocytes in monolayer culture appear elongated and spindle-shaped (FIG. 3, left panel).

The level of de novo proteoglycan synthesis, assessed by ³⁵SO₄ incorporation, indicated that cells propagated on microcarriers produce ECM rich in ³⁵SO₄-containing proteoglycans with MW>10,000. The estimated size of the newly synthesized and exported proteoglycans, based on SDS-PAGE, was about 270 kDa, and appeared as a diffuse and faint band.

Phenotypic expression, using types I and II collagen and aggrecan as markers, of chondrocytes retrieved from different tissue sources using monolayer and spinner culture methods. The gene expression displayed by chondrocytes obtained directly from the tissue (Po), chondrocytes propagated in monolayer culture for one passage about 2 weeks (M) and chondrocytes propagated in microcarreir spinner culture (Sp) was analyzed by RT-PCR. FIG. 4 shows the RT-PCR profiles that indicate that cells from articular knee cartilage of OA patients, ankle cartilage and nasal septal cartilage maintain expression of type II collagen and aggrecan. In contrast, type I collagen is down-regulated.

The expression of two other collagen molecules i.e., types IX and XI were also analyzed in articular chondrocytes obtained directly from knee cartilage (Po), chondrocytes propagated in monolayer culture for one passage for about 2 weeks, chondrocytes propagated in microcarrier spinner culture for about 2 weeks (Sp). The RT-PC profiles shown in FIG. 5 show that expression of type IX collagen is enhanced when chondrocytes were propagated in microcarrier spinner culture.

Cells cultured on microcarriers immunostain more intensely for type II collagen and for proteoglycans (aggrecan, keratan and chondroitin sulfate) than chondrocytes from monolayer culture (data not shown). In contrast, staining for type I collagen was negligible for cells in microcarriers but intense in monolayer cultures.

When propagated in microcarrier spinner culture, the chondrocyte-seeded microcarriers aggregate as shown in FIG. 6 taken on day 7. Over time, this aggregation continues, resulting in formation of cartilage-like tissue that continues to increase in size as shown in FIG. 7 on day 60.

Because of the observed similarities in behavior of chondrocytes cultured on microcarriers to chondrocytes in cartilage tissue, the term “cartilage analog” applies to cell-microcarrier aggregates cultured for approximately 2 weeks to produce a cartilage-like construct. Specific methods for culturing chondrocytes on microcarrier particles to produce cartilage analogs are also described in U.S. patent application Ser. No. 09/825,632.

### EXAMPLE 2

### Preparation of Bone Analogs

Human trabecular bone-derived osteoblasts were isolated by collagenase digestion from tissue discarded following hip or knee reconstruction. The cells were seeded at 4×10³/cm² in monolayer culture and propagated in HY medium (Hank's Balanced Salt Solution, HBSS+10% Fetal Calf Serum) until nearly confluent (about 2 weeks). Cells were harvested by trypsinization and seeded onto collagen microcarriers (4×10³/cm² on Cellagen® beads, ICN, Cleveland, Ohio). The cultures (P3) were incubated for 5 to 15 days at 37° C., 5% CO₂. After 15 days, osteoblasts were recovered from microcarrier cultures and were reseeded (4×10³/cm²) onto microcarriers for 5 to 15 days (P4). These P3 or P4 osteoblast-microcarrier-ECM aggregates are referred to as “bone analogs”.

After the culture period, the supernatant was removed and saved in microcentrifuge tubes for osteocalcin determination. DNA synthesis rates were determined by labeling the cells with 1 μCi/well of ³H-thymidine during the last 24 hours of incubation. For Western blots, cells were resuspended in lysis buffer containing protease inhibitors. Protein concentrations were determined by using the DC Protein Assay kit (Bio-Rad; Hercules, Calif.). Total cell extracts (20 μg) were electrophoresed on 7 % SDS-polyacrylamide gel overlaid with stacking gel. The proteins were transferred from gel to nitrocellulose paper (Bio-Rad; Hercules, Calif.) using a Mini-trans-blot electrophoresis transfer cell (Bio-Rad). Type I collagen was immunolocalized using monoclonal type I collagen antibody (Oncogene Research Products; Boston, Mass.) linked to a goat-anti mouse IgG (Bio-Rad). Collagen was visualized by reaction of the conjugated alkaline phosphatase (1:3000 dilution) with the Immun-star Chemiluminescent substrate solution (Bio-Rad). To visualize the bands, the membrane blot was exposed to BioMax Light Film(VWR, NJ). The results were analyzed using the STAT® 6.0 program with multiple comparison, one-way ANOVA. Statistical significance was set at P<0.05.

Propagation of trabecular bone cells on collagen microcarriers enhanced DNA synthesis as indicated by greater radiolabeled thymidine uptake compared to monolayer cultures (P<0.05). Cells retrieved from microcarriers (P3) and reseeded onto another set of microcarriers (P4) also exhibited increased proliferative capacity compared to matched monolayer cultures (P<0.05). However, both subsequent passages (P4) showed lower DNA synthetic rates. Osteocalcin levels in spinner cultures were also higher in spinner (30 ng/10⁵cells) than monolayer cultures (10 ng/10⁵ cells) on day 5. In contrast, osteocalcin levels measured on day 15 were variable. Synthesis of collagen type I was also more pronounced in microcarrier cultures detected by Western blots.

### EXAMPLE 3

### Preparation of Mesenchyme Analogs

Human Mesenchymal Stem Cells (hMSCs) were harvested by trypsinization from 4 flasks. 4.0 million hMSCs were seeded into a spinner culture reactor containing 1000 cm² of microcarrier (Cellagen) surface area (4 thousand cells per cm²) in 120 ml of hMSC culture medium. On days 7 and 14, 10 ml of suspension was withdrawn from the spinner culture and frozen. Cell counts from spinner cultures on days 7 and 14 were 5.3 and 12.8 thousand cells per cm², respectively. HMSCs on some microcarrier beads were nearly confluent by day 7. RT-PCR on samples from spinner cultures from day 14 was consistent with expected hMSC profiles (positive for type I collagen and aggrecan; negative for type II collagen). Day 14 aggregates are suitable as Mesenchyme Analogs.

### EXAMPLE 4

### Use of the Cartilage Tissue Analog System for Drug Screening.

Chondrocytes comprise less than 10% of the total cartilage volume, but they produce and break down macromolecules that make up this tissue. The processes that control chondrocyte synthesis and degradation of cartilage components in health and disease are regulated by a wide variety of factors that are biological, chemical, physical and mechanical in nature. Chondrocytes recognize and respond to: (a) biological factors such as growth and differentiation factors, hormones, cytokines, chemokines, radicals; (b) chemical agents and drugs; (c) physical factors such as heat, oxygen tension; and (d) mechanical stimuli such as compression, shear and strain. However, the responsiveness of chondrocytes as they are localized in their physiologic three-dimensional cartilaginous millieu may not be the same when analyzed as isolated cells in vitro.

Chondrocyte-microcarrier cartilage analogs respond to biological, chemical and physical stimuli in a similar fashion as when they are localized in the extracellular matrix (ECM) of cartilage. Responses to these factors can be identified by changes in their proliferative capacity, metabolism and phenotype expression. Changes in chondrocyte phenotypic expression may be indicated by collagen and proteoglycan expression at the message and protein level. Products that chondrocytes synthesize such as cytokines or enzymes may regulate the cartilage-ECM structure and integrity. Chondrocyte-microcarrier constructs respond to growth factors such as TGF-β, anti-inflammatory drugs such as NSAIDS (ibuprofen), dietary supplements and botanical products, cytokines (TNF-α, IL-1α or IL-1β), oxygen tension, and mechanical stimuli such as shear and fluid flow.

A preliminary experiment was conducted to analyze the effect of agents such as growth factors (TGF-β₁) and non-steroidal anti-inflammatory drugs (NSAIDS; Ibuprofen). Human chondrocytes from donors undergoing nasal septal defect surgery were isolated by enzymatic digestion. The cells were seeded onto microcarriers at approximately 4×10³ cells/cm², and incubated in spinner flasks at 60 rpm and at 37° C. and 5% CO₂ until the cells reach confluency at about 14 days. At this point, the chondrocytes produce extracellular matrix-like products consisting of type II collagen, high-molecular-weight proteoglycans and aggrecan. The constructs also start to form microaggregates. These aggregates are referred to here as “cartilage analogs”.

Equivalent aliquots of these analogs were transferred to six 25-ml spinner flasks. Aliquots (5 ml) of control medium (no additive), or medium containing human platelet TGF-β₁ (10 ng/ml; Sigma Chemical Co., St. Louis, Mo.) or Ibuprofen (140 μg/ml; Sigma) were added to duplicate flasks. TGF-β₁ is a growth factor known to stimulate proteoglycan synthesis in chondrocyte cultures [2] and cartilage explant cultures, while Ibuprofen is an anti-inflammatory drug known to inhibit proteoglycan synthesis. The concentrations of TGF-β₁ and Ibuprofen were selected based on earlier studies demonstrating the effect of these concentrations on isolated chondrocytes or cartilage explants. The cultures were incubated for two days. 24 hours prior to terminating the cultures, 300 μCi of ³⁵S—Na₂SO₄ (NEN Life Science Products, Boston, Mass.) was added to each flask to measure incorporation into proteoglycans during the last 24 hours of culture.

Following termination of the cultures, cells were pelleted by centrifugation and proteoglycans were extracted from the supernatant medium and the cell fraction with 4 M guanidine HCl for 24 hours at 4° C. Guanidine HCl extracts were dialyzed extensively against deionized class I water using 6,000 Da cut-off dialysis tubing (The Spectrum Companies, Gardena, Calif.). Post-dialysis volumes were measured and triplicate 100 μl samples were counted in a LS-6500 Liquid Scintillation Counter (Beckman Instruments, Inc., Palo Alto, Calif.) using Cytoscint-ES Cocktail (ICN Research Products, Costa Mesa, Calif.). The total ³⁵SO₄ incorporation was expressed as cpm per 10⁶ cells. Another set of dialyzed samples were dried on a Speed-Vac (Savant, Inc., Farmingdale, N.Y.) and was then reconstituted in sample buffer consisting of 1% sodium dodecylsulfate, 30% sucrose and 0.025% brophenol blue. The samples were boiled for 5 min and then loaded on an 0.75% agarose gel in electrophoresis buffer. After electrophoresis, the gel was treated with Entensify (NEN Life Science Products), dried and autoradiographed at −70° C.

Cells were fixed with cytospin collection fluid (Shandon Lipshaw, Pittsburgh, Pa.) and stained with either Diff-Quick (Baxter Healthcare Products, Miami, Fla.) or with the respective antibodies for immuno-cytochemistry. A set of slides was incubated with monospecific antibodies against chondroitin-4-sulfate or keratan (ICN). Immunostaining for proteoglycans was visualized using the immunoperoxidase technique with diaminobenzidine as substrate, yielding a brownish colored precipitate. Cell preparations were counterstained with 0.5% toluidine blue. Human chondrocytes at passage 1 served as the positive cell control for proteoglycans. Negative cell controls for proteoglycans consisted of human lymphocytes. Specificity of the immunoperoxidase staining was verified by substituting media or an irrelevant anti-human immunoglobulin antibody for antibodies to proteoglycans. There was no staining when the primary antibody was omitted nor when antibody to human immunoglobulin was used to replace antibodies against proteoglycans.

Chondrocytes harvested from spinner culture on day 14 stained for unsulfated chondroitin, chondroitin-4-sulfate, chondroiting-6-sulfate and keratan sulfate. The same pattern of homogeneous intracellular immunostaining for proteoglycans was observed in chondrocytes cultured in control growth medium alone after the 2-day test period. More intense immunostaining for proteoglycans was noted in chondrocyte cultures incubated with TGF-β₁ for two days, while cells exposed to Ibuprofen showed more faint immunostaining compared to control cells. The stimulatory effect of TGF-β₁ on proteoglycan synthesis was verified by higher sulfate incorporation, which was detectable in both the cell-associated (non-secreted) and media-associated (secreted) fractions, when compared to controls (P<0.05). In contrast, exposure to Ibuprofen decreased the cell-associated sulfate incorporation. In all cases, approximately 99% of proteoglycans synthesized during the test period were secreted and detected in the supernatant fraction.

Based on this preliminary experiment, a subsequent study investigated the effect of a proposed chondroprotective drug (PPS5, a pentosan poly-phosphate). De novo proteoglycan synthesis was used as a marker for chondrocytic activity. Using the procedures described above, the cartilage tissue-analog system again reflected the expected effects of these agents (FIG. 8). Ibuprofen again decreased proteoglycan synthesis whereas TGF-β₁ again enhanced protoglycan synthesis during the assay period. In this study, the test drug PPS5 failed to shown any significant effect on proteoglycan synthesis. These preliminary results demonstrate an approach for using the cartilage analog system for in vitro screening of drug candidates using known cartilage biology modulators as positive and negative control agents.

### EXAMPLE 5

### In Vitro Testing Using Cartilage Tissue Analog in a 96-Well Assay Format.

Nasal chondrocytes were obtained from a patient undergoing septal reconstruction and then propagated in monolayer culture. Chondrocytes were retrieved from monolayer culture at passage 2 (about 2 weeks in culture) by trypsinization. Cells were washed and pelleted by centrifugation. Chondrocytes (4×10⁶) were then seeded in a spinner flask onto 1000 cm² of Cellagen® microcarrier beads in 120 ml of HY Media [1]. The spinner flask was placed on a spinner culture plate at 60 RPM until 90% confluence (about 10 days). The cell-seeded microcarriers were then sedimented and the volume of media was reduced to 6 ml. Aliquots (200 μl) were transferred into a total of 30 wells in a 96-well plate. Control media alone (100 μl) or media containing FGF-9 (10 ng/ml ) and heparin (2 μg/ml) was added to the wells. The plate was incubated on a platform shaker (Innova 200, New Brunswick Scientific) at 115 rpm for 24 hrs, 37° C., 5%CO₂. FIG. 9 shows the chondrocyte-seeded microcarriers at about 90% confluence when placed in the 96-well plates. After 24 hours, control chondrocytes incubated in HY media alone had 1-1.4×10⁶ cells per well and 400-500 ng of DNA per well (FIG. 10). Chondrocytes incubated with the growth factor FGF-9 showed significantly higher cell counts (1.3-1.6×10⁶, P<0.05) and DNA (˜600 ng) than cells incubated in control media. Chondrocytes incubated with FGF-9 also showed significantly higher levels of total RNA (P<0.01; FIG. 11). Phenotypic analysis by RT-PCR (FIG. 12) indicated that FGF-9 enhanced expression of type II collagen but not type I. The proliferation marker cyclin D1 (Cyc D1) was also enhanced in chondrocytes upon exposure to FGF-9. The phenotypic marker aggrecan and Cyclin CDk4 were not altered. The housekeeping genes GAPDH and S14 confirmed equal loading. FGF-9 also increased the concentration of newly synthesized and secreted proteoglycans (P<0.05; FIG. 13). These studies suggest that exposure to FGF-9 for 24 hours modulated proliferative indices and phenotype expression of nasal chondrocytes. These studies also demonstrate that the 96-well micro-cartilage analog assay can be adapted to evaluate the effect of mitogenic or differentiation factors such as FGF-9 on cartilage analogs.

### EXAMPLE 6

### Use of Culture Insert System in 96-Well Culture Plates.

The inventors envision that cartilage analogs can be cultured as described in Example 5 when placed in individual semipermeable holders that are inserted into the wells of 96-well microtiter plates as shown in FIG. 1. FGF-9 can be added at the same final concentrations as used in Example 5. Control media alone can be added to separate wells as controls. The culture systems can then be placed on a rocking platform for two days of additional culture in an incubator at 37° C. and 5% CO₂. The inserts containing the cartilage analogs then can be separated from the microtiter plate, and the analogs can be retrieved for immediate analysis or cryopreserved for later analysis. The spent tissue culture medium also can be used for immediate analysis or frozen for subsequent analysis. Outcome measures to determine the effect of the test agent on the viability, proliferative capacity, phenotypic expression, synthesis of chondrocyte products such as cytokines can then be evaluated as described in Example 5.

### EXAMPLE 7

### Use of the Bone Analogs for Screening.

The inventors envision that tissue analogs can be used for high-throughput screening of libraries of compounds for drug candidates or for biocompatibility testing. For example, bone analogs can be prepared as described in Example 2 and then transferred to the wells of a 96-well cell culture plate. Culture of the bone analogs can continue in 4-6 of the wells in standard growth medium, while the bone analogs in other wells can be exposed to individual or mixtures of candidate drugs to determine the effects of the candidate drugs on bone cell viability, proliferation, matrix secretion or initiation of mineralization. After culturing the two analogs for 7, 14 or 21 days, the analogs from each well can be collected by sedimentation and processed to measure bone markers such as type I collagen, osteocalcin or osteopontin, or MRNA for these or other markers by RT-PCR as described in Example 2. The most effective drug candidates can thereby be determined based on their ability to promote cell proliferation, matrix secretion or mineralization capacity.

### EXAMPLE 8

### Use of Cartilage Analogs for Genomic Screening.

The inventors envision that tissue analogs can be used for searching for genes that are regulated during normal cell growth, or following exposure to cell-modifying factors such as drug candidates, potential toxins, tissue extracts, body fluids or extracts, enzymes or enzyme inhibitors, antibodies, antigens, growth factors, cytokines, integrins, hormones, differentiation factors or mitogens. Genes or gene products identified by such procedures might then, for example, be the targets for new drugs or for gene therapy.

For example, cartilage analogs can be prepared by the methods in Example 1, then split into two aliquots and transferred to two spinner culture vessels. Culture of the cartilage analogs can continue with the first vessel in standard growth medium, while the cartilage analogs are exposed to standard growth medium containing TNF-α to promote an arthritis-like condition. After culturing the two vessels for 2, 4 or 7 days, the cartilage analogs from each vessel can be collected by centrifugation and processed to isolate cDNA from the two populations of cartilage analogs. Differential cDNA displays between the cDNA of the two populations will reveal genes that are differentially up-regulated or down-regulated during exposure to TNF-α. Such genes can be further tested to determine if they are potential targets for new anti-arthritis drugs.

### EXAMPLE 9

### Use of Stem Cell Mesenchyme Analogs for Screening for Differentiation Factors.

The inventors envision that tissue analogs can be used for screening for factors that are regulate or modify normal cell growth following exposure of the tissue analogs to cell-modifying factors such as drug candidates, potential toxins, tissue extracts, body fluids or extracts, enzymes or enzyme inhibitors, antibodies, growth factors, cytokines, integrins, hormones, differentiation factors or mitogens. Factors identified by such procedures might then, for example, be candidates for therapeutic drugs.

For example, mesenchyme analogs can be prepared by the methods in Example 3, then transferred to the wells of a 96-well cell culture plate. Culture of the mesenchyme analogs can continue in 4-6 of the wells in standard growth medium, while the mesenchyme analogs in other wells are exposed to standard growth medium containing TGF-β₁ to initiate differentiation of the mesenchyme into chondrogenesis. After culturing the two analogs for 7, 14 and 21 days, the analogs from each well can be collected by sedimentation and processed to measure cartilage markers such as secreted type II collagen or proteoglycans such as aggrecan, or MRNA for these markers by RT-PCR. The most effective concentration of TGF-β₁ for producing rapid chondrogenesis in mesenchyme analogs can thereby be determined.

### LITERATURE CITED

1. Frondoza C, Sohrabi A, Hungerford D: Human chondrocytes proliferate and produce matrix components in microcarrier suspension culture. Biomaterials 1996; 17:879-888.
2. Redini, F, Min W, et al.: Differential expression of membrane-anchored proteoglycans in rabbit articular chondrocytes cultured in monolayers and in alginate beads. Effect of transforming growth factor-beta 1. Biochimica Biophysica Acta. 1997; 1355(1): 20-32.
3. Adkisson H D, Maloney W J, el al: Scaffold-independent Neocartilage Formation: A novel approach to cartilage engineering. Transactions of the Orthopaedic Research Society 1998; Paper 803.
4. Sung H J, Adkisson H D, et al.: Cytokine-mediated down-regulation of BMP expression in human neocartilage. Transactions Orthopaedic Research Society 2001; Paper 0098.

1. A test system comprising tissue analogs composed of aggregates of cells, microcarrier beads and extracellular matrix in a cell culture compartment.

2. The test system of claim 1 wherein the tissue analog is prepared using chondrocytes.

3. The test system of claim 1 wherein the cells are selected from the group of chondrocytes, osteoblasts, fibroblasts derived from skin, tendon, ligament, meniscus, disk, or synovium, mesenchymal stem cells, pluripotent stem cells derived from bone marrow stroma, muscle, skin, fat, periosteum or perichondrium, embryonic stem cells, or combinations of these cells.

4. The test system of claim 1 wherein the tissue analog is prepared using microcarrier beads selected from the group of inorganic materials including calcium phosphates, calcium carbonates, calcium sulfates, glasses or combinations of these materials; biopolymers including collagen, gelatin, chitin, chitosan or chitosan derivatives, fibrin, dextran, agarose, or calcium alginate; particles of tissues including bone or demineralized bone, cartilage, tendon, ligament, fascia, intestinal mucosa, or chemically modified derivatives of these materials; or synthetic polymeric materials including polylactic acid, polyglycolic acid or combinations of the two, polyurethanes, polycarbonates, polyacrylates, or polypeptides; or combinations of said inorganic materials, biopolymers, particles of tissues, or synthetic polymers.

5. The test system of claim 1 wherein the tissue analog is prepared using microcarrier beads in the size range of 50-1000 μm in diameter.

6. The test system of claim 1 wherein the tissue analog is prepared using microcarrier beads in the range of 100-300 μm in diameter.

7. The test system of claim 1 wherein the analogs are prepared in a suspension or “spinner” culture system.

8. The test system of claim 1 wherein the cell culture compartment is a standard culture vessel.

9. The test system of claim 1 wherein the cell culture compartment is a suspension or spinner culture vessel.

10. The test system of claim 1 wherein the cell culture compartment is a well of a standard multi-well culture plate.

11. The test system of claim 1 wherein the tissue analog is cryopreserved in the cell culture compartment for storage or transportation.

12. The test system of claim 1 wherein the tissue analog is an aggregate of cells, microcarrier particles and extracellular matrix that express a disease-like condition.

13. The test system of claim 1 wherein the tissue analog is an aggregate of chondrocytes, microcarrier particles and extracellular matrix that have been treated with TNF-α and/or IL-1 to produce an arthritis-like condition.

14. The test system of claim 1 in combination with an agent to be tested.

15. The method of conducting a test comprising preparing tissue analogs by culturing cells to grow and proliferate on microcarrier particles for an extended period of time, thereby producing cell-microcarrier aggregates, exposing the tissue analog to chemical or physical test stimuli in a cell culture compartment, and observing and recording the result.

16. The method of claim 15 wherein the tissue analog is prepared using chondrocytes.

17. The method of claim 15 wherein the cells are selected from the group of chondrocytes, osteoblasts, fibroblasts derived from skin, tendon, ligament, meniscus, disk, or synovium, mesenchymal stem cells, pluripotent stem cells derived from bone marrow stroma, muscle, skin, fat, periosteum or perichondrium, embryonic stem cells, or combinations of these cells.

18. The method of claim 15 wherein the tissue analog is prepared using microcarrier beads selected from the group of inorganic materials including calcium phosphates, calcium carbonates, calcium sulfates, glasses or combinations of these materials; biopolymers including collagen, gelatin, chitin, chitosan or chitosan derivatives, fibrin, dextran, agarose, or calcium alginate; particles of tissues including bone or demineralized bone, cartilage, tendon, ligament, fascia, intestinal mucosa, or chemically modified derivatives of these materials; or synthetic polymeric materials including polylactic acid, polyglycolic acid or combinations of the two, polyurethanes, polycarbonates, polyacrylates, or polypeptides; or combinations of said inorganic materials, biopolymers, particles of tissues, or synthetic polymers.

19. The method of claim 15 wherein the tissue analog is prepared using microcarrier beads in the size range of 50-1000 μm in diameter.

20. The method of claim 15 wherein the tissue analog is prepared using microcarrier beads in the range of 100-300 μm in diameter.

21. The method of claim 15 wherein the analogs are prepared in a suspension or “spinner” culture system.

22. The method of claim 15 wherein the cell culture compartment is a standard culture vessel.

23. The method of claim 15 wherein the cell culture compartment is a suspension or spinner culture vessel.

24. The method of claim 15 wherein the cell culture compartment is a well of a standard multi-well culture plate.

25. The method of claim 15 wherein the tissue analog is cryopreserved in the cell culture compartment for storage or transportation.

26. The method of claim 15 wherein the tissue analog is an aggregate of cells, microcarrier particles and extracellular matrix that expresses a disease-like condition.

27. The method of claim 15 wherein the tissue analog is an aggregate of chondrocytes, microcarrier particles and extracellular matrix that have been treated with TNF-α and/or IL-1 to produce an arthritis-like condition.

28. The method of claim 15 wherein the chemical stimulus is a drug candidate, potential toxin, tissue extract, body fluid or extract, enzyme or enzyme inhibitor, antibody, antigen, growth factor, cytokine, integrin, hormone, differentiation factor or mitogen.

29. The method whereby a tissue analog is used to perform an in vitro test comprising the steps of
1. culturing cells to grow and produce extracellular matrix on microcarrier particles for an extended period of time, thereby producing cell-microcarrier bead aggregate tissue analogs;
2. placing the tissue analog in culture medium in a cell culture compartment for culturing the analogs;
3. introducing the desired test substance into the culture medium; and
4. determining the effect of the test substance on the tissue analog by monitoring the outcomes compared to a test standard.

30. The method of claim 29 wherein the tissue analog is prepared using chondrocytes.

31. The method of claim 29 wherein the cells are selected from the group of chondrocytes, osteoblasts, fibroblasts derived from skin, tendon, ligament, meniscus, disk, or synovium, mesenchymal stem cells, pluripotent stem cells derived from bone marrow stroma, muscle, skin, fat, periosteum or perichondrium, embryonic stem cells, or combinations of these cells.

32. The method of claim 29 wherein the tissue analog is prepared using microcarrier beads selected from the group of inorganic materials including calcium phosphates, calcium carbonates, calcium sulfates, glasses or combinations of these materials; biopolymers including collagen, gelatin, chitin, chitosan or chitosan derivatives, fibrin, dextran, agarose, or calcium alginate; particles of tissues including bone or demineralized bone, cartilage, tendon, ligament, fascia, intestinal mucosa, or chemically modified derivatives of these materials; or synthetic polymeric materials including polylactic acid, polyglycolic acid or combinations of the two, polyurethanes, polycarbonates, polyacrylates, or polypeptides; or combinations of said inorganic materials, biopolymers, particles of tissues, or synthetic polymers.

33. The method of claim 29 wherein the tissue analog is prepared using microcarrier beads in the size range of 50-1000 μm in diameter.

34. The method of claim 29 wherein the tissue analogs are prepared using microcarrier beads in the range of 100-300 μm in diameter.

35. The method of claim 29 wherein the tissue analogs are prepared in a suspension or “spinner” culture system.

36. The method of claim 29 wherein the cell culture compartment is a standard culture vessel.

37. The method of claim 29 wherein the cell culture compartment is a suspension or spinner culture vessel.

38. The method of claim 29 wherein the cell culture compartment is a well of a standard multi-well culture plate.

39. The method of claim 29 wherein the tissue analog is cryopreserved in the cell culture compartment for storage or transportation.

40. The method of claim 29 wherein the tissue analog is an aggregate of cells, microcarrier particles and extracellular matrix that expresses a disease-like condition.

41. The method of claim 29 wherein the tissue analog is an aggregate of chondrocytes, microcarrier particles and extracellular matrix that have been treated with TNF-α and/or IL-1 to produce an arthritis-like condition.

42. The method of claim 29 wherein the test substance is a drug candidate, potential toxin, tissue extract, body fluid or extract, enzyme or enzyme inhibitor, antibody, antigen, growth factor, cytokine, integrin, hormone, differentiation factor or mitogen.

43. The method of claim 29 wherein the outcome of the test is analyzed using an assay of tissue analog function.

44. The method of claim 29 in which the outcome of the in vitro test is analyzed by measuring cell viability, cell number or proliferative capacity, proteoglycan synthesis by ³⁵ SO₄ incorporation, RNA analysis by Reverse Transcriptase-Polymerse Chain Reaction (RT-PCR), cytological-immunocytochemical analysis of extracellular matrix products or secreted cellular products, Western blots, Enzyme Linked Immuno Sorbent Assays (ELISA), determination of enzyme products, or gene profiles using microarray techniques.

45. The method of claim 29 wherein the outcome of the in vitro test is analyzed continuously, periodically or after a specific period of time.

46. A kit for performing a test comprising tissue analogs formed by culturing cells to grow and proliferate on microcarrier particles for an extended period of time, thereby producing cell-microcarrier aggregates, which are then dispersed in the wells of a multi-well cell culture plate.

47. The kit of claim 46 wherein the tissue analog is prepared using chondrocytes.

48. The kit of claim 46 wherein the cells are selected from the group of chondrocytes, osteoblasts, fibroblasts derived from skin, tendon, ligament, meniscus, disk, or synovium, mesenchymal stem cells, pluripotent stem cells derived from bone marrow stroma, muscle, skin, fat, periosteum or perichondrium, embryonic stem cells, or combinations of these cells.

49. The kit of claim 46 wherein the tissue analog is prepared using microcarrier beads selected from the group of inorganic materials including calcium phosphates, calcium carbonates, calcium sulfates, glasses or combinations of these materials; biopolymers including collagen, gelatin, chitin, chitosan or chitosan derivatives, fibrin, dextran, agarose, or calcium alginate; particles of tissues including bone or demineralized bone, cartilage, tendon, ligament, fascia, intestinal mucosa, or chemically modified derivatives of these materials; or synthetic polymeric materials including polylactic acid, polyglycolic acid or combinations of the two, polyurethanes, polycarbonates, polyacrylates, or polypeptides; or combinations of said inorganic materials, biopolymers, particles of tissues, or synthetic polymers.

50. The kit of claim 46 wherein the tissue analog is prepared using microcarrier beads in the size range of 50-1000 μm in diameter.

51. The kit of claim 46 wherein the tissue analog is prepared using microcarrier beads in the range of 100-300 μm in diameter.

52. The kit of claim 46 wherein the analogs are prepared in a suspension or “spinner” culture system.

53. The kit of claim 46 wherein the tissue analog is cryopreserved in the cell culture well for storage or transportation.

54. The kit of claim 46 wherein the tissue analog is an aggregate of cells, microcarrier particles and extracellular matrix that express a disease-like condition.

55. The kit of claim 46 wherein the tissue analog is an aggregate of chondrocytes, microcarrier particles and extracellular matrix that have been treated with TNF-α and/or IL-1 to produce an arthritis-like condition.

56. The kit of claim 46 along with instructions for use.

57. The kit of claim 46 in combination with an agent to be tested.

58. A device for contacting and separating cell-microcarrier bead tissue analogs from culture medium in multi-well culture plates comprising an insert device that fits into a standard multi-well culture plate, wherein the bottoms of the insert wells consist of a semipermeable membrane or screen allowing rapid diffusion or convection of solutes and fluids between the wells of the insert and the multi-well culture plate, and wherein tissue analogs are cultured, and wherein the tissues analogs are retained in the insert wells when the culture plate and insert device are separated.

59. The test system of claim 1 wherein the cell culture compartment is the well of an insert device that fits into a standard multi-well culture plate, wherein the bottoms of the insert wells consist of a semipermeable membrane or screen allowing rapid diffusion or convection of solutes and fluids between the wells of the insert and the microtiter plate, and wherein tissue analogs are cultured, and whereby the tissues analogs are retained in the insert wells when the culture plate and insert device are separated.

60. The method of claim 15 for conducting a test wherein the cell culture compartment is the well of an insert device that fits into a standard multi-well culture plate, wherein the bottoms of the insert wells consist of a semipermeable membrane or screen allowing rapid diffusion or convection of solutes and fluids between the wells of the insert and the microtiter plate, and wherein tissue analogs are cultured, and whereby the tissues analogs are retained in the insert wells when the culture plate and insert device are separated.

61. The method of claim 29 whereby a tissue analog is used to perform a test wherein the cell culture compartment is the well of an insert device that fits into a standard multi-well culture plate, wherein the bottoms of the insert wells consist of a semipermeable membrane or screen allowing rapid diffusion or convection of solutes and fluids between the wells of the insert and the microtiter plate, and wherein tissue analogs are cultured, and whereby the tissues analogs are retained in the insert wells when the culture plate and insert device are separated.

62. The kit of claim 46 for performing a test wherein the multi-well culture plate is combined with an insert device that fits into the culture plate, wherein the bottoms of the insert wells consist of a semipermeable membrane or screen allowing rapid diffusion or convection of solutes and fluids between the wells of the insert and the microtiter plate, and wherein tissue analogs are cultured, and whereby the tissues analogs are retained in the insert wells when the culture plate and insert device are separated.
